(19) United States
(12) Patent Application Publication
(10) Pub. No.: US 2021/0353501 A1
(43) Pub. Date: Nov. 18, 2021

(54) HYDROPHOBICALLY-MODIFIED POLYMER FOAMS AND METHODS OF USE

(71) Applicant: MEDCURA, INC., Riverdale, MD (US)

(72) Inventor: Matthew DOWLING, Riverdale, MD (US)

(21) Appl. No.: 16/498,896

(22) PCT Filed: Apr. 2, 2018
(86) PCT No.: PCT/US2018/025742

§ 371 Date: Sep. 27, 2019

Related U.S. Application Data
(60) Provisional application No. 62/479,903, filed on Mar. 31, 2017.

(51) Int. Cl. A61J 1/20; A61L 26/00; C08L 5/08

(52) U.S. Cl. CPC A61J 1/2096; A61L 26/0085; A61L 2400/04; C08L 5/08; A61L 26/0004; A61L 26/0023

(57) ABSTRACT

The present invention relates, in part, to hydrophobically-modified polymers and foams and their use in the control of bleeding.

### PRIORITY

This application claims the benefit of U.S. Provisional Application No. 62/479,903, filed Mar. 31, 2017, the contents of which are hereby incorporated by reference in its entirety.

### BACKGROUND

Bleeds can range from small, superficial bleeds to those caused by traumatic injuries, hematological disorders, and invasive medical procedures, among others. When the site of bleeding is not readily accessible, such as inside a body cavity, hemorrhage control within the body cavity is difficult to achieve and can lead to many complications. For example, uncontrolled bleeding remains the leading cause of death in the battlefield.

Accordingly, this is a need for compositions and methods that provide effective hemostatic control, including for bleeds that are not easily accessible with hemostatic devices. The present invention addresses these needs and other objectives.

### SUMMARY

In various aspects and embodiments, the present invention provides hemostat products and methods of treating bleeds, including cavity bleeds or other difficult to access bleeds, including nose bleeds (epistaxis).

In various embodiments, the invention provides a device having at least two compartments each containing a releasable, flowable product. The first compartment comprises a solution of an acidified hydrophobically-modified polymer that gels or self-assembles in the presence of blood. A second compartment comprises a solution of a bicarbonate or carbonate salt at an alkaline pH. Delivery of the contents to a bleed results in the production of a stable foam that can quickly control bleeds, including bleeds that are difficult to access, and/or that are not easily amenable to compression.

In some embodiments, the container is any container that allows for controlled release of the contents, such as a syringe. In some embodiments, the product comprises a double-barrel syringe comprising two compartments and a static mixing tip. The static mixing tip stabilizes carbon dioxide (CO₂) gas and allows for direct application of a hydrophobically modified-polymer foam.

The first compartment of the container contains a modified polymer, such as hydrophobically modified chitosan. In some embodiments, the modified polymer is modified with C8 to C20 hydrophobic grafts (which can be linear hydrocarbon grafts present along the backbone of the polymer. In the case of hydrophobically-modified chitosan, about 10% to about 50% of available functional groups along the backbone may be occupied by the hydrophobic grafts. In some embodiments, the hm-chitosan includes C12 grafts as well as some grafts larger than C12 to provide added integrity to the foam. In some embodiments, from 5% to about 40% of the grafts are C16 or C18. In some embodiments, the modified polymer (e.g., hm-chitosan) may be present in the first container at about 0.1 to about 2% by weight of the solution in the first compartment, such as from 0.2 to 0.8% by weight in some embodiments. The modified polymer may be formulated at a pH of from 2.5 to 4.5, and in some embodiments from pH 3 to pH 4.5. In some embodiments, the modified polymer is dissolved in acetic acid, lactic acid, ascorbic acid, or citric acid. For example, in the case of acetic acid and hm-chitosan, the acetic acid may be from 0.3 to 0.5M.

The second compartment of the container comprises a solution of a carbonate or bicarbonate salt, such as sodium bicarbonate. In some embodiments, the bicarbonate solution is from 0.2 to 0.5M. The bicarbonate or carbonate solution may be at pH 8 to 10, or from about pH 8 to about pH 9. In some embodiments, second compartment further comprises one or more negatively charged polymers, which in some embodiments are hydrophobically modified (e.g., hydrophobically modified alginate).

Upon mixing of the first compartment and the second compartment using the mixing tip, an expanding foam is generated due to stabilization of bubbles formed from the generation of CO₂ gas. In various embodiments, the foam is stable for at least 10 minutes, is stable when mixed with blood, and allows for transfer of gases in some embodiments.

As disclosed herein, a product of the present disclosure may be used in methods related to treating bleeds, including cavity bleeds (e.g., epistaxis), by releasing the contents of the product onto the site of bleeding. For example, the bleeding wound may be epistaxis and the contents of the container may be released into one or more nostrils. In some embodiments, the resulting foam or gel is applied to the naval cavity through a conical applicator, or is attached to a substrate that is removed from the nostril once the foam/gel is applied.

Other aspects and embodiments of the invention will be apparent from the following detailed description.

### DESCRIPTION OF THE FIGURES

FIG. 1A illustrates a dual barrel syringe for creating Hm-chitosan foam. Hm-chitosan is solubilized in acetic acid in Side A. Upon delivery, HmChitosan will be mixed with sodium bicarbonate from Side B.

FIG. 1B illustrates production of a stable foam upon generation of CO₂ gas.

FIG. 2 is a graph showing surface tension measurements for Hm-chitosan with varying modification degree with C12 hydrophobic grafts.

FIG. 3 is a graph showing a representative frequency sweep for Hm-chitosan foam and solution. Circles represent 0.3 wt % Hm-chitosan hybrid in 0.04M AcOH/0.15M NaAcetate. Squares represent a pure solution of the same formulation. Final concentrations are reported.

FIG. 4 is a graph showing storage modulus for foam mixtures. Foam and blood (or PBS) were mixed at a 1:1 ratio by weight. Pre-mix foam formulation was 0.3 wt % Hm-chitosan hybrid in 0.04M AcOH/0.15M NaAcetate. Storage modulus (and loss modulus, not shown) are preserved when mixing foam with blood. Significant decrease in moduli observed when mixing with PBS (foam is obliterated).

FIG. 5 is a graph showing a time sweep at 1 Hz for HmChitosan foam. Foam formulation: 0.3 wt % Hm-chitosan hybrid in 0.040M AcOH/0.15M NaAcetate.

FIG. 6 is a graph showing a representative strain sweep for 0.3 wt % Hm-chitosan hybrid in 0.04M AcOH/0.15M NaAcetate foam.

FIG. 7 illustrates the overall experimental set up for the in vivo rat anterior epistaxis model

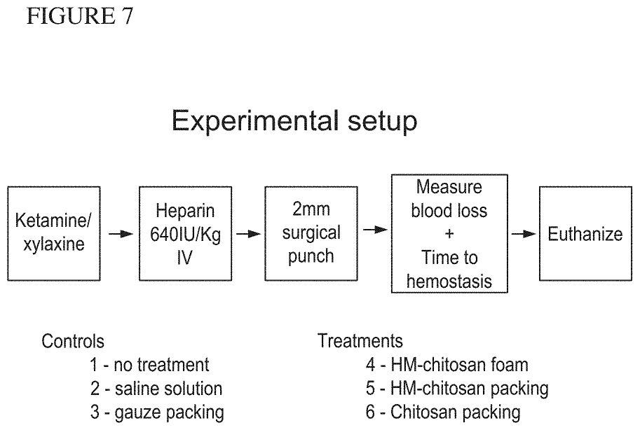

FIG. 8 is an image showing the saline/no treatment control for the experiment.

FIG. 9 is an image showing the HM-chitosan gauze packing treatment for the experiment.

FIG. 10 is a graph showing the results of average blood loss in the experiment for different treatments, including the control gauze and the no treatment control.

FIG. 11 is a graph showing average cumulative blood loss for different treatments and controls.

FIG. 12 is a line graph showing the results of blood loss per minute post nasal punch.

FIG. 13 is a graph showing the results of the time between the first and last blood droplet.

### DETAILED DESCRIPTION

In various aspects and embodiments, the present invention provides hemostat products and methods of treating bleeds, including cavity bleeds or other difficult to access bleeds, including nose bleeds (epistaxis). In various embodiments, the invention provides a device having at least two compartments each containing a releasable, flowable product. The first compartment comprises a solution of an acidified hydrophobically-modified polymer that gels or self-assembles in the presence of blood. A second compartment comprises a solution of a bicarbonate or carbonate salt at an alkaline pH. Delivery of the contents to a bleed results in the production of a stable foam that can quickly control bleeds, including bleeds that are difficult to access, and/or that are not easily amenable to control by compression. In accordance with embodiments of the invention, the foams are stable for at least 10 minutes (e.g., do not immediately dissipate) and remain stable in the presence of blood.

In some embodiments, the container is any container that allows for controlled release of the contents, such as a syringe. The ratio of volume released in various embodiments can be from 1:0.5 to 0.5:1 of the first and second containers. In some embodiments, the ratio of the solution in the first compartment and the ratio of the solution in the second compartment are about 1:1. In some embodiments, the product comprises a double-barrel syringe comprising two compartments and a static mixing tip. The static mixing tip stabilizes carbon dioxide (CO₂) gas and allows for direct application of a hydrophobically modified foam.

The first compartment of the container contains a modified polymer, such as modified chitosan. In some embodiments, the biopolymer is hydrophobically-modified chitosan. The modified polymer (e.g., hm-chitosan) may be present in the first container at 0.1 to about 2% by weight, or about 0.1 to about 1% by weight, or about 0.2 to about 0.8% by weight (e.g., about 0.5-0.6% by weight) of the solution in the first compartment. The modified polymer may be formulated at a pH of from 2.5 to 4.5, and in some embodiments is preferably in the range of pH 3 to pH 4.5. In some embodiments, the modified polymer is dissolved in acetic acid, lactic acid, ascorbic acid, or citric acid. For example, in the case of acetic acid and hm-chitosan, the acetic acid may be from 0.3 to 0.5M, or in some embodiments, from 0.35 to 0.45 M. In some embodiments, lactic acid is used to avoid offensive odor.

The second compartment of the container comprises a solution of a carbonate or bicarbonate salt, such as sodium bicarbonate. In the second compartment, the bicarbonate solution may be around 0.1 wt % to about 1 wt %, or from about 0.2 wt % to about 0.5 wt %. In some embodiments, the bicarbonate solution is from 0.2 to 0.5M, such as from about 0.2 to about 0.4 M, or about 0.3M. The bicarbonate or carbonate solution may be at pH 8 to pH 10, or from about pH 8 to about pH 9. In some embodiments, the modified polymer is placed in the second compartment. For example, hydrophobically modified alginate can be placed in the second compartment. In other embodiments employing hm-chitosan in the first compartment, the second compartment may further comprise a negatively charged polymer, which may improve the integrity of the resulting foam. Exemplary negative charged polymers include alginate, pectin, dextran, carboxymethylcellulose, or xanthan gum. In some embodiments, the negatively charged polymer is hydrophobically modified (e.g., hydrophobically-modified alginate).

Upon mixing of the first compartment and the second compartment using the mixing tip, an expanding foam is generated due to stabilization of bubbles formed from the generation of CO₂ gas. In general, a smaller bubble size yields a longer drainage time, and therefore, longer stability. Foam stability (i.e., ‘foamability’) can be adjusted based on hydrophobic modification degrees and types, and also on the hydrophobically modified polymer's molecular weight. In addition, the static mixer foams of the present disclosure are able to form in situ, such that foam may be generated within the blood. As demonstrated in the experiments herein, the storage modulus for the foam mixtures is preserved when mixed with blood but not with PBS. This may be a demonstration of the interaction between blood cells and the modified polymer, such as gelation or aggregation.

As disclosed herein, a product of the present disclosure may be used in methods related to treating a bleeds, including cavity bleeds (e.g., epistaxis), by releasing the contents of the product onto the site of bleeding. For example, the bleeding wound may be epistaxis and the contents of the container may be released into one or more nostrils. In some embodiments, the resulting foam or a gel is applied to the naval cavity through a conical applicator, or is attached to a substrate that is removed from the nostril once the foam/gel is applied.

An exemplary hm-polymer material for use in accordance with embodiments of the invention is chitosan. Chitosan is the common name of the linear, random copolymer that consists of β-(1-4)-linked D-glucosamine and N-acetyl-D-glucosamine. The molecular structure of chitosan consists of a linear backbone linked with glycosidic bonds. Chitosan is the major component of crustacean shells such as crab, shrimp, krill and crawfish shells. Additionally, chitosan is the second most abundant natural biopolymer after cellulose. Commercial chitosan samples are typically prepared by chemical de-N-acetylation of chitin under alkaline conditions. Depending on the source of the natural chitin (extracted from shells) and its production process, chitosan can differ in size (average molecular weight Mw) and degree of N-acetylation (% DA). While the poor solubility of chitosan in water and in common organic solvents restricts its applications, reactive amino groups in the chitosan backbone make it possible to chemically conjugate chitosan with various molecules and to modulate its properties for use as a hemostat product.

In various embodiments, the degree of deacetylation of chitin may range from about 40-100%, or in some embodiments, from 60 to 100%, or from 40 to about 90%, or from 50 to about 80%, which determines the charge density. The structure of chitosan (deacetylated) is depicted in Formula 1:

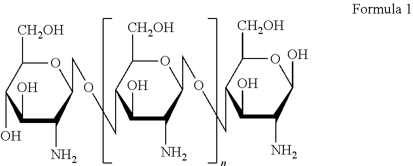

These repeating monomeric units include a free amino group, which makes molecules or compounds containing chitosan or its derivatives readily reactive. The hydrophobic modification of the chitosan backbone is through the association of an amphiphilic compound with the amino group, such that the hydrophobic tail of the amphiphilic compound is bound with the hydrophilic backbone structure.

The polymer that forms the backbone is chitosan, or similar polymer of synthetic or natural origin, including for example, water-soluble polysaccharides and water-soluble polypeptides. In some embodiments, the polymer is one or more hm-polysaccharides, including but not limited to cellulosics, chitosans and alginates, all of which are abundant, natural biopolymers. In some embodiments, the hm-biopolymer contains cationic groups.

The natural origin of these polysaccharides varies, cellulosics are found in plants, whereas chitosans and alginates are found in the exoskeleton or outer membrane of a variety of living organisms. Many of these naturally occurring polymers, in addition to being able to form long stable chains for forming the polymer backbone, have properties that are beneficial for a hemostat application, including anti-microbial properties.

In some embodiments, the hm-chitosan is derived from a deacteylated chitin, which may be derived from one or more of crab, shrimp, krill, and crawfish.

The form of the natural polymers used may vary to include standard states, derivatives and other various formulations. For example, the hm-cellulosics may be formed from, without limitation, hydroxyethyl cellulose, hydroxypropyl cellulose, methyl cellulose, hydroxypropyl methyl cellulose, and/or hydroethyl methyl cellulose. Hm-chitosans may be prepared from, without limitation, the following chitosan salts: chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof. Hm-alginates may be prepared from, without limitation, sodium alginate, potassium alginate, magnesium alginate, calcium alginate, and/or aluminum alginate. It is to be understood that various other forms of any of these natural polysaccharides that provide the proper functional capabilities may be employed without departing from the scope and spirit of the present invention.

In some embodiments, the polymeric component is a mixture of polysaccharides. For instance, the mixture may be of various different sub-classes of a single polymer class. Alternatively, the mixture may include two or more different classes of polymer, for instance a chitosan and an alginate.

In various embodiments, the biopolymer is an hm-chitosan, which may be prepared from a chitosan having a degree of deacetylation of from about 40 to about 90%, or from about 50 to about 80%, or from about 60% to about 75%. The polymer may have 10% to about 50% of functional groups occupied by a hydrophobic group. In some embodiments, the degree of substitution of the hydrophobic substituent on the biopolymer is from about 1 to about 100 moles of the hydrophobic group per mole of the polymer. In some embodiments, the degree of substitution of the hydrophobic substituent on the biopolymer is from about 5 to about 100 moles of the hydrophobic group per mole of the polymer. In some embodiments, the degree of substitution of the hydrophobic substituent on the biopolymer is from about 5 to about 50 moles of the hydrophobic group per mole of the polymer. In some embodiments, the degree of substitution of the hydrophobic substituent on the biopolymer is from about 10 to about 50 moles of the hydrophobic group per mole of the polymer. In some embodiments, the degree of substitution of the hydrophobic substituent on the biopolymer is from about 10 to about 30 moles of the hydrophobic group per mole of the polymer. In some embodiments, the degree of substitution of the hydrophobic substituent on the polysaccharide is from about 40 to 65 moles of the hydrophobic substituent per mole of the polysaccharide. In some embodiments, the degree of substitution of the hydrophobic substituent on the polysaccharide is from about 1 to 30 moles of the hydrophobic substituent per mole of the polysaccharide. In some embodiments, the molecular weight of the polysaccharides used as the biopolymer range from about 25,000 to about 1,500,000 grams per mole. In various embodiments, the molecular weight of the biopolymer ranges from about 40,000 to about 500,000 grams per more, or from about 50,000 to about 250,000 grams per mole, or from about 50,000 to about 100,000 grams per mole. As used herein, the term “molecular weight” means weight average molecular weight. Methods for determining average molecular weight of bio-polymers include low angle laser light scattering (LLS) and Size Exclusion Chromatography (SEC). In performing low angle LLS, a dilute solution of the polysaccharide, typically 2% or less, is placed in the path of a monochromatic laser. Light scattered from the sample hits the detector, which is positioned at a low angle relative to the laser source. Fluctuation in scattered light over time is correlated with the average molecular weight of the polysaccharide in solution. In performing SEC measurements, again a dilute solution of biopolymer, typically 2% or less, is injected into a packed column. The polysaccharide is separated based on the size of the dissolved polymer molecules and compared with a series of standards to derive the molecular weight.

A hydrophobically modified biopolymer material for incorporation into a container having at least two compartments can be an aqueous formulation. In some embodiments, the hydrophobically-modified chitosan is present at about 0.1% to about 2% by weight, or from about 0.1 to about 1% of the total weight of the solution. In some embodiments, the hydrophobically-modified chitosan is present at about 0.2% to about 0.8% by weight relative to the total weight of the solution. In some embodiments, the hydrophobically-modified chitosan is present at 0.3% by weight to about 0.7% by weight (e.g., about 0.5% or about 0.6% by weight).

Hydrophobic moieties can be independently selected from saturated hydrocarbons (e.g., alkanes), which are optionally acyl groups, and unsaturated hydrocarbons (e.g., alkenes, alkynes), which may be linear, branched or cyclic. In some embodiments, the hydrophobic moieties include aromatic and/or polyaromatic hydrocarbons. In some embodiments, the hydrophobic moiety is a hydrocarbon having from about 4 to about 100 carbon atoms, or from about 8 to about 60 carbon atoms, or from about 8 to about 28 carbon atoms, or from about 8 to about 36 carbons, or from about 8 to about 18 carbon atoms, or from about 8 to about 20 carbon atoms. The hydrophobic groups may be linear hydrocarbons having about 12 to about 18 carbon atoms on average, such as an average of from 14 to 16 carbon atoms on average.

In some embodiments, the hydrophobically modified chitosan contains linear, saturated C8, C10, or C12 hydrocarbon moieties, which provide good foaming properties. In some embodiments, the integrity of the foam is improved with addition of from 5% to about 40% (relative to the total number of hydrophobic grafts) of C16 or C18 linear, hydrocarbon moieties. In some embodiments, the hydrophobically-modified chitosan has from 10% to about 30% C16 or C18 hydrocarbon moieties. In some embodiments, the hydrophobic moieties are C12 and C18, where the majority are C12.

The hydrophobic substituents may be a hydrocarbon group having from about 8 to about 18 carbon atoms attached to the backbone of the one biopolymer, and in some embodiments comprises an alkyl group. In some embodiments, the hydrocarbon group comprises an arylalkyl group. As used herein, the term “arylalkyl group” means a group containing both aromatic and aliphatic structures.

The hemostat product may comprise numerous hydrophobically modified biopolymer compounds. These compounds comprise a biopolymer (such as chitosan) backbone that includes a hydrophilically reactive functional group (e.g., amino groups) that binds with the hydrophilically reactive head groups (e.g., carbonyl functional group) of an amphiphilic compound (e.g., aldehyde), to form the hm-chitosan or other hm-polymer. The head group is further associated with a hydrophobic tail group. In the current embodiment, the hydrophobic tail may be, for example, a hydrocarbon. Thus, a hydrophobic tail is associated with the biopolymer backbone providing the hydrophobic modification to the molecule that extends from the backbone and may interact with a surrounding environment in numerous ways, such as through hydrophobic interaction with materials.

Examples of procedures for modifying polymers are as follows.

Alginates can be hydrophobically modified by exchanging their positively charged counterions (e.g. Na+) with tertiary-butyl ammonium (TBA) ions using a sulfonated ion exchange resin. The resulting TBA-alginate is dissolved in dimethylsulfoxide (DMSO) where reaction occurs between alkyl (or aryl) bromides and the carboxylate groups along the alginate backbone. Alginate can also be modified by fatty amine groups (e.g. dodecyl amine), followed by addition of 1-ethyl-3-(3-dimethylaminopropyl) carbodiimide, via EDC coupling.

Cellulosics can be hydrophobically modified by first treating the cellulosic material with a large excess highly basic aqueous solution (e.g. 20 wt % sodium hydroxide in water). The alkali cellulose is then removed from solution and vigorously mixed with an emulsifying solution (for example, oleic acid) containing the reactant, which is an alkyl (or aryl) halide (e.g. dodecyl bromide).

Chitosans can be hydrophobically modified by reaction of alkyl (or aryl) aldehydes with primary amine groups along the chitosan backbone in a 50/50 (v/v) % of aqueous 0.2 M acetic acid and ethanol. After reaction, the resulting Schiff bases, or imine groups, are reduced to stable secondary amines by dropwise addition of the reducing agent sodium cyanoborohydride.

The degree of substitution of the hydrophobic substituent on the polymer is up to 50% of available functional groups, for example, amines in the case of chitosan. For example, the hydrophobic substituent can be added to from 10 to 50% of available amines, or from 20 to 50% of available amine, or from 30 to 50% of available amines. It is contemplated that more than one particular hydrophobic substituent may be substituted onto the polymer, provided that the total substitution level is substantially within the ranges set forth above.

In some embodiments, the hydrophobic substituent is derived from an amphiphilic compound, meaning it is composed of a hydrophilic Head group and a hydrophobic Tail group. The Head group binds with the polymer and positions the Tail group to extend from the backbone of the polymer scaffold. This makes the hydrophobic Tail group available for hydrophobic interactions. The Tail group is a hydrocarbon of various forms.

Hydrocarbons that find use in accordance with this disclosure may be classified as saturated hydrocarbons, unsaturated hydrocarbons, and aromatic hydrocarbons. From this basic classification system there exist many derivatives and further types of compounds that build therefrom. For example, numerous and varied compounds include more than one aromatic ring and are generally referred to as polyaromatic hydrocarbons (PAH). In some embodiments, the hydrophobic moiety is aliphatic. Aliphatic compounds, carbon atoms can be joined together in straight chains, branched chains, or rings (in which case they are called alicyclic). They can be joined by single bonds (alkanes), double bonds (alkenes), or triple bonds (alkynes). Besides hydrogen, other elements can be bound to the carbon chain, the most common being oxygen, nitrogen, sulfur, and chlorine. Those of ordinary skill in the art will recognize that other molecules may also be bound to the carbon chains and that compounds of such heteroatomic structure are contemplated as falling within the scope of the current invention.

The hydrophobic Tail group of the amphiphilic compound bound to the polymer backbone of the current invention is capable of branching and/or allowing the inclusion of side chains onto its carbon backbone. It may be understood that the strength of the hydrophobic interaction is based upon the available amount of “hydrophobes” that may interact amongst themselves or one another. Thus, it may further promote the hydrophobic effect by increasing the amount of and/or hydrophobic nature of the hydrophobic Tail group that is interacting. For instance, a hydrophobic Tail group, which in its original form may include a hydrocarbon chain, may promote an increase in its hydrophobicity (ability to hydrophobically bond and strength of hydrophobic interaction) by having a hydrophobic side chain attach to one of the carbons of its carbon backbone.

In some embodiments, the current invention contemplates the use of various molecules and/or compounds that may increase hemorrhage control, durability, water repellent properties, and/or flexibility of the hemostat product. The side chains may be linear chains, aromatic, aliphatic, cyclic, polycyclic, or any various other types of hydrophobic side chains as contemplated by those skilled in the art.

In some embodiments, the hydrophobic grafts include an alicyclic, cycloalkane, or cycloalkene. For example, the hydrophobic group may be both aliphatic and cyclic with or without side chains attached. In some embodiments, the cyclic groups are carbocyclic groups, which may be saturated or unsaturated (aromatic or non-aromatic).

In some embodiments, the hydrophobic grafts include aromatic hydrocarbon, or polycyclic aromatic hydrocarbon, or heterocyclic moieties. Heterocyclic groups may include, in addition to carbon, at least one atom such as nitrogen, oxygen, or sulfur, as part of the ring. Examples include pyridine (C₅H₅N), Pyrimidine (C₄H₄N₂) and Dioxane.

Some of the contemplated hydrophobic side chains may include the following:

TABLE 1
Linear Alkanes

| Number of | | Common |
|---|---|---|
| C Atoms | Formula | Name |
| 1 | CH₄ | Methane |
| 2 | C₂H₆ | Ethane |
| 3 | C₃H₈ | Propane |
| 4 | C₄H₁₀ | n-Butane |
| 5 | C₅H₁₂ | n-Pentane |
| 6 | C₆H₁₄ | n-Hexane |
| 7 | C₇H₁₆ | n-Heptane |
| 8 | C₈H₁₈ | n-Octane |
| 9 | C₉H₂₀ | n-Nonane |
| 10 | C₁₀H₂₂ | n-Decane |
| 11 | C₁₁H₂₄ | n-Undecane |
| 12 | C₁₂H₂₆ | n-Dodecane |
| 13 | C₁₃H₂₈ | n-Trideacane |
| 14 | C₁₄H₃₀ | n-Tetradecane |
| 15 | C₁₅H₃₂ | n-Pentadecane |
| 16 | C₁₆H₃₄ | n-Hexadecane |
| 17 | C₁₇H₃₆ | n-Heptadecane |
| 18 | C₁₈H₃₈ | n-Octadecane |
| 19 | C₁₉H₄₀ | n-Nonadecane |
| 20 | C₂₀H₄₂ | n-Eicosane |
| 21 | C₂₁H₄₄ | n-Heneicosane |
| 22 | C₂₂H₄₆ | n-Docosane |
| 23 | C₂₃H₄₈ | n-Tricosane |
| 24 | C₂₄H₅₀ | n-Tetracosane |
| 25 | C₂₅H₅₂ | n-Pentacosane |
| 26 | C₂₆H₅₄ | n-Hexacosane |
| 27 | C₂₇H₅₆ | n-Heptacosane |
| 28 | C₂₈H₅₈ | n-Octacosane |
| 29 | C₂₉H₆₀ | n-Nonacosane |
| 30 | C₃₀H₆₂ | n-Triacontane |
| 31 | C₃₁H₆₄ | n-Hentraiacontane |
| 32 | C₃₂H₆₆ | n-Dotriacontane |
| 33 | C₃₃H₆₈ | n-Tritriacontane |
| 34 | C₃₄H₇₀ | n-Tetratriacontane |
| 35 | C₃₅H₇₂ | n-Pentatriacontane |
| 36 | C₃₆H₇₄ | n-Hexatriacontane |
| 37 | C₃₇H₇₆ | n-Heptatriacontane |
| 38 | C₃₈H₇₈ | n-Octatriacontane |
| 39 | C₃₉H₈₀ | n-Nonactriacontane |
| 40 | C₄₀H₈₂ | n-Tetracontane |
| 41 | C₄₁H₈₄ | n-Hentatetracontane |
| 42 | C₄₂H₈₆ | n-Dotetracontane |
| 43 | C₄₃H₈₈ | n-Tritetracontane |
| 44 | C₄₄H₉₀ | n-Tetratetracontane |
| 45 | C₄₅H₉₂ | n-Pentatetracontane |
| 46 | C₄₆H₉₄ | n-Hexatetracontane |
| 47 | C₄₇H₉₆ | n-Heptatetracontane |
| 48 | C₄₈H₉₈ | n-Octatetracontane |
| 49 | C₄₉H₁₀₀ | n-Nonatetracontane |
| 50 | C₅₀H₁₀₂ | n-Pentacontane |
| 51 | C₅₁H₁₀₄ | n-Henpentacontane |
| 52 | C₅₂H₁₀₆ | n-Dopentacontane |
| 53 | C₅₃H₁₀₈ | n-Tripentacontane |
| 54 | C₅₄H₁₁₀ | n-Tetrapentacontane |
| 55 | C₅₅H₁₁₂ | n-Pentapentacontane |
| 56 | C₅₆H₁₁₄ | n-Hexapentacontane |
| 57 | C₅₇H₁₁₆ | n-Heptapentacontane |
| 58 | C₅₈H₁₁₈ | n-Octapentacontane |
| 59 | C₅₉H₁₂₀ | n-Nonapentacontane |
| 60 | C₆₀H₁₂₂ | n-Hexacontane |
| 61 | C₆₁H₁₂₄ | n-Henhexacontane |
| 62 | C₆₂H₁₂₆ | n-Dohexacontane |
| 63 | C₆₃H₁₂₈ | n-Trihexacontane |
| 64 | C₆₄H₁₃₀ | n-Tetrahexacontane |
| 65 | C₆₅H₁₃₂ | n-Pentahexacontane |
| 66 | C₆₆H₁₃₄ | n-Hexahexacontane |
| 67 | C₆₇H₁₃₆ | n-Heptahexacontane |
| 68 | C₆₈H₁₃₈ | n-Octahexacontane |
| 69 | C₆₉H₁₄₀ | n-Nonahexacontane |
| 70 | C₇₀H₁₄₂ | n-Heptacontane |
| 71 | C₇₁H₁₄₄ | n-Henheptacontane |
| 72 | C₇₂H₁₄₆ | n-Doheptacontane |
| 73 | C₇₃H₁₄₈ | n-Triheptacontane |
| 74 | C₇₄H₁₅₀ | n-Tetraheptacontane |
| 75 | C₇₅H₁₅₂ | n-Pentaheptacontane |
| 76 | C₇₆H₁₅₄ | n-Hexaheptacontane |
| 77 | C₇₇H₁₅₆ | n-Heptaheptacontane |
| 78 | C₇₈H₁₅₈ | n-Octaheptacontane |
| 79 | C₇₉H₁₆₀ | n-Nonaheptacontane |
| 80 | C₈₀H₁₆₂ | n-Otcacontane |
| 81 | C₈₁H₁₆₄ | n-Henoctacontane |
| 82 | C₈₂H₁₆₆ | n-Dooctacontane |
| 83 | C₈₃H₁₆₈ | n-Trioctacontane |
| 84 | C₈₄H₁₇₀ | n-Tetraoctacontane |
| 85 | C₈₅H₁₇₂ | n-Pentaoctacontane |
| 86 | C₈₆H₁₇₄ | n-Hexaoctacontane |
| 87 | C₈₇H₁₇₆ | n-Heptaoctacontane |
| 88 | C₈₈H₁₇₈ | n-Octaoctacontane |
| 89 | C₈₉H₁₈₀ | n-Nonaoctacontane |
| 90 | C₉₀H₁₈₂ | n-Nonacontane |
| 91 | C₉₁H₁₈₄ | n-Hennonacontane |
| 92 | C₉₂H₁₈₆ | n-Dononacontane |
| 93 | C₉₃H₁₈₈ | n-Trinonacontane |
| 94 | C₉₄H₁₉₀ | n-Tetranonacontane |
| 95 | C₉₅H₁₉₂ | n-Pentanonacontane |
| 96 | C₉₆H₁₉₄ | n-Hexanonacontane |
| 97 | C₉₇H₁₉₆ | n-Heptanonacontane |
| 98 | C₉₈H₁₉₈ | n-Octanonacontane |
| 99 | C₉₉H₂₀₀ | n-Nonanonacontane |
| 100 | C₁₀₀H₂₀₂ | n-Hectane |
| 101 | C₁₀₁H₂₀₄ | n-Henihectane |
| 102 | C₁₀₂H₂₀₆ | n-Dohectane |
| 103 | C₁₀₃H₂₀₈ | n-Trihectane |
| 104 | C₁₀₄H₂₁₀ | n-Tetrahectane |
| 105 | C₁₀₅H₂₁₂ | n-Pentahectane |
| 106 | C₁₀₆H₂₁₄ | n-Hexahectane |
| 107 | C₁₀₇H₂₁₆ | n-Heptahectane |
| 108 | C₁₀₈H₂₁₈ | n-Octahectane |
| 109 | C₁₀₉H₂₂₀ | n-Nonahectane |
| 110 | C₁₁₀H₂₂₂ | n-Decahectane |
| 111 | C₁₁₁H₂₂₄ | n-Undecahectane |

TABLE 2
Cyclic Compounds

| | | Example |
|---|---|---|
| Polycyclic Compounds | Sub-Types | Compounds |
| Bridged | Bicyclo compound | adamantine |
| Compound - compounds | | amantadine |
| which contain | | biperiden |
| interlocking rings | | memantine |
| | | methenamine |
| | | rimantadine |
| Macrocyclic | Calixarene | |
| Compounds | Crown Compounds | |
| | Cyclodextrins | |
| | Cycloparaffins | |
| | Ethers, Cyclic | |
| | Lactans, macrocyclic | |
| | Macrolides | |
| | Peptides, Cyclic | |
| | Tetrapyrroles | |
| | Trichothecenes | |
| Polycyclic | Acenaphthenes | |
| Hydrocarbons, | Anthracenes | |
| Aromatic | Azulenes | |
| | Benz(a)anthracenes | |
| | Benzocycloheptenes | |
| | Fluorenes | |
| | Indenes | |
| | Naphthalenes | |
| | Phenalenes | |
| | Phenanthrenes | |
| | Pyrenes | |
| | Spiro Compounds | |
| Steroids | Androstanes | |
| | Bile Acids and Salts | |
| | Bufanolides | |
| | Cardanolides | |
| | Cholanes | |
| | Choestanes | |
| | Cyclosteroids | |
| | Estranes | |
| | Gonanes | |
| | Homosteroids | |
| | Hydroxysteroids | |
| | Ketosteroids | |
| | Norsteroids | |
| | Prenanes | |
| | Secsteroids | |
| | Spirostans | |
| | Steroids, Brominated | |
| | Steroids, Chlorinated | |
| | Steroids, Fluorinated | |
| | Steroids, Heterocyclic | |

Any aspect or embodiment described herein can be combined with any other aspect or embodiment as disclosed herein.

Other aspects and embodiments of the invention will be apparent to the skilled artisan from this disclosure.

The invention will be further described in the following examples, which do not limit the scope of the invention described in the claims.

### EXAMPLES

### Example 1: Designing hm-chitosan Foams

To establish a robust foam to treat blood loss (e.g., epistaxis), a dual-barrel syringe with a static mixing tip was used to stabilize carbon dioxide (CO₂) gas with hydrophobically-modified chitosan. CO₂ is generated by the reaction between acetic acid and sodium bicarbonate:

CH₃COOH+NaHCO₃→CH₃COONa+CO_{2(g)}+H₂O

Since this foam system is intended for use as a hemostat (e.g., for epistaxis), it is important to minimize the acid content and maintain an appropriate salt concentration.

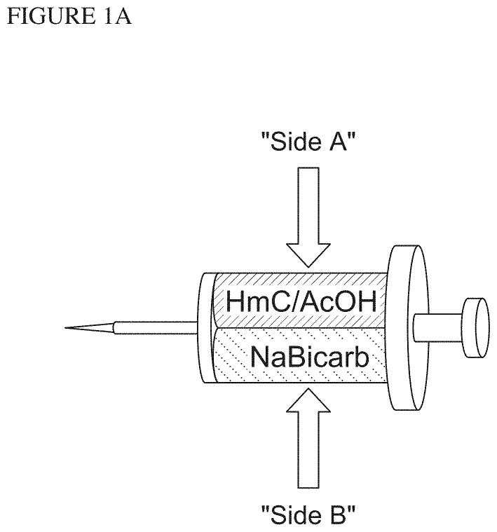

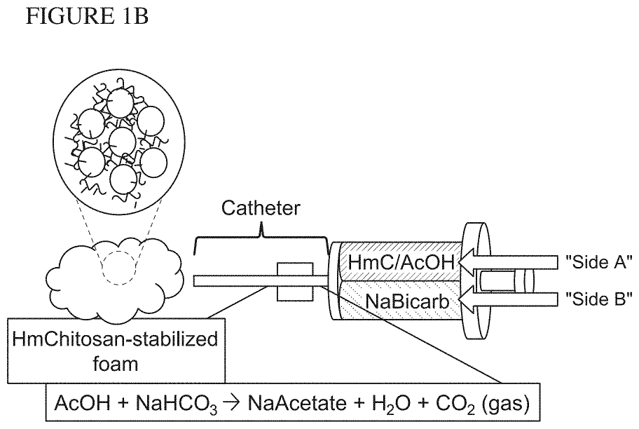

An image of the syringe system is shown in FIG. 1A and FIG. 1B. In side A, HmC is dissolved in AcOH, in this case 0.38M AcOH; side B contains an equal volume of 0.3M NaBicarb. In the examples of this disclosure, the formulations are specific to a 1:1 syringe barrel ratio with 2×8 mm tip (#4B19; manufacturer: PlasPak; as distributed by DeDoes Inc). Other tip sizes and syringe volume/mix ratios can be employed. For example, 2 mL 0.38M AcOH mixed with 2 mL 0.3M NaBicarb theoretically produces 7.46 mL CO₂ gas, and the aqueous phase contains 0.15M sodium acetate (NaAcetate) and 0.04M AcOH leftover. Accordingly, the resultant salt concentration was enough to osmotically balance the natural salt content of blood plasma (0.15M NaCl), and the final acid content was under 0.05M, which was determined to be appropriate for blood, since when mixed with blood, the sample remained bright red over long periods of time rather than turning dark, which would have indicated a lower level of oxygen in the blood. An approximation of the final pH of the aqueous phase of the foam can be calculated from pKa values for AcOH and NaAcetate; in the experiments shown, the pH was 5.3.

Hm-Chitosan generally requires an acidic environment to dissolve. HmAlginate (HmA) does not have this issue, and thus potentially could be included in side B. For hm-chitosan, the pH of side A should be larger than pH 3 to avoid precipitate. Further, the viscosity of the solutions must be sufficiently low to allow operative syringing and foam formation.

### Example 2: Characterization of Static Mixer Foams

This example analyzes the mechanical properties of the hm-chotosan foam.

Generally, the bulk modulus of the foam is primarily affected by the enclosed gas phase while the shear modulus is dictated by surface tension and bubble size. The bulk modulus can be probed via dilatation; as each dilation provides information regarding the adsorption/desorption of surfactants. Shear rheology probes the interaction between surfactants. Additionally, bubble size, or the surface tension/bubble size ratio, also contributes to the shear modulus; however, surface tension, surface elasticity (surface elasticity can be measured using a rheometer with special geometry), and viscosity can also each affect bubble size. In general, a smaller bubble size yields a longer drainage time, and therefore, longer stability. The capillary number is a property that relates contributions from the viscous forces and surface tension. The hmpolymers disclosed herein seem likely to most resemble proteins, in that they have both hydrophobic and hydrophilic components and are large molecules that have self-assembly properties. Surface tension of proteins increases with increasing molecular weight, however small, disordered, flexible proteins are known to be good surfactants. Moreover, the effect of salts in hmpolymer solutions affect viscosity and surface tension by screening electrostatic repulsions allowing for a more compact molecular structure. These are some of the key mechanical properties of foam.

Other key parameters for foam formation (i.e., foamability) include foam capacity (FC), maximum foam density (MD), percent volumetric foam stability (% FVS) and percent liquid stability (% FLS), each are presented below. For clarification, V_{foam0} is the volume of formed foam, V_{gas} is the volume of gas introduced for the formation of the foam, V_{liq0} is the volume of liquid entrapped in the formed foam, and V_{foam} and V_{liq} are the volumes of foam and entrapped liquid at time t, respectively. These are qualitative parameters to describe foams, as represented by the following formulas:

FC=V_{foam0}/V_{gas}

MD=V_{liq0}/V_{foam0}

% FVS=(V_{foam}/V_{foam0})×100

% FLS=(V_{liq}/V_{liq0})×100

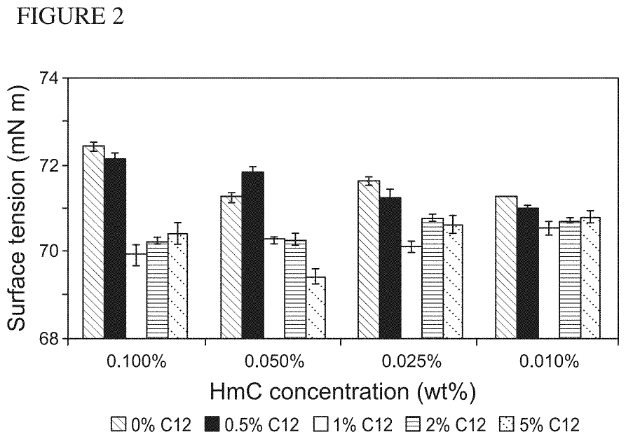

Based on the above overview of foam properties, one would hypothesize that the decrease in surface tension caused by a surfactant has a significant impact on foamability. However, based on the preliminary surface tension measurements completed for Hm-alginate and Hm-chitosan, the decrease in surface tension appeared to be inadequate to stabilize gas. As shown in FIG. 2, the lowest surface tension measured is about 69 mNm, where pure water is 72.5 and pure acetone is 23.5. To determine whether this was a discrepancy in the surface tension measurement technique, or whether it was the actual surface tension measurement, dynamic measurements were conducted with a hanging drop tensiometer (i.e., Fisher Scientific Surface Tensiometer 21 with a bent platinum ring, which was able to measure pure water and acetone within 5% accuracy).

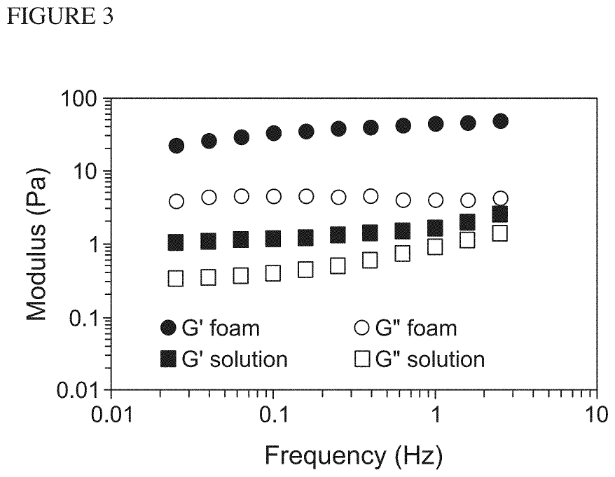

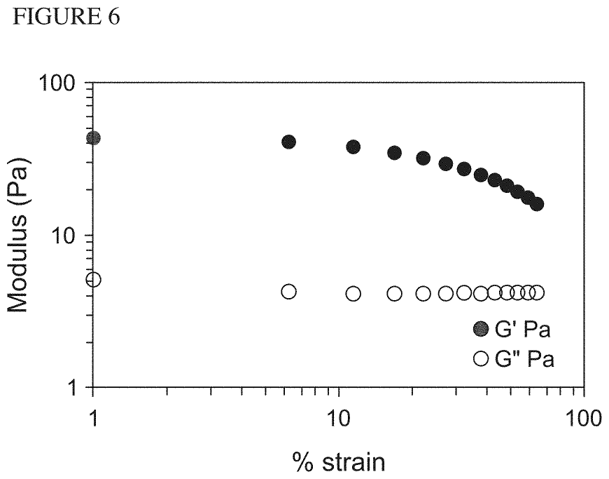

Typical mechanical behavior of foams follows that of Bingham plastics, where elastic deformation at low strain leads into plastic deformation (from bubble rearrangement) and flow. Under flow, it is possible for bubbles to rearrange and segregate by size. Foam stability is controlled by drainage, bubble coalescence, and bubble disproportionation. For the static mixer foams stabilized with Hm-chitosan, typical dynamic rheological behavior is shown in FIG. 3. FIG. 3 shows that the foam exhibits gel-like behavior compared to the pure solution of an equivalent composition. FIG. 6 shows shear thinning behavior at a strain of about 10%.

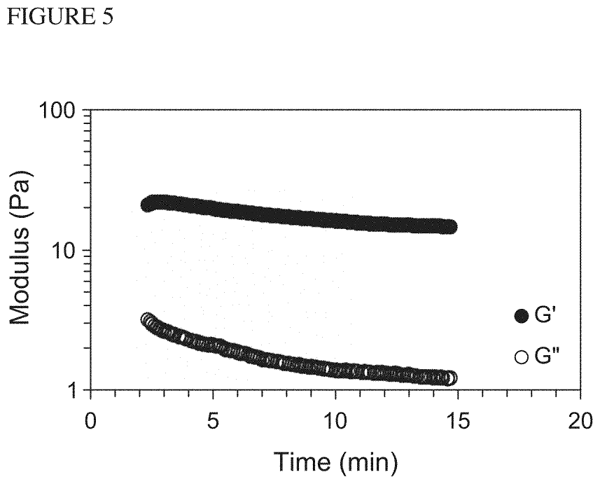

Monitoring foam height, or shear modulus, over time allows for an indication of foam stability. FIG. 5 shows a time sweep for an HmC foam where the storage modulus remains fairly constant over 15 minutes, whereas the loss modulus decreases by about half after about 10 minutes. A decrease in G″ with steady G′ may be indicative of bubble coalescence and drainage, as the overall liquid content decreases and bubble size increases.

Presented in this example are results for Hm-chitosan hybrid (5% C12 1% C18) using a double-barrel syringe with a static mixing tip to generate a foam from the reaction between AcOH and NaBicarb.

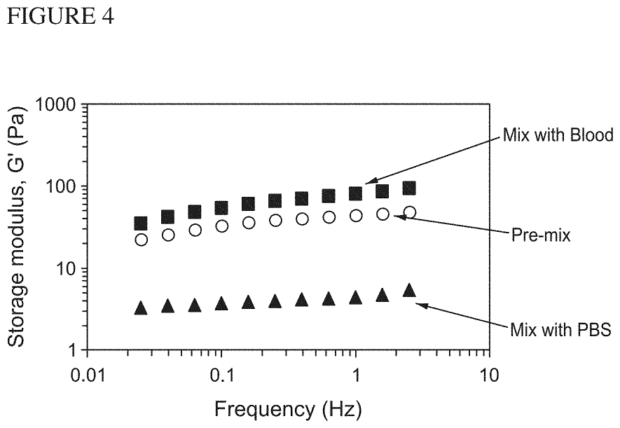

Regarding the mixture of foams with blood, the physical manipulation of a foam changes the number of bubbles as well as size. With this in view, the static mixer foams of the present disclosure are able to form in situ, allowing the formation of the foam within a blood sample. This may be a way to create foam/blood mixtures without compromising the natural foam structure. FIG. 4 shows that the G′ is preserved when mixing the foam with blood and not when mixed with PBS. This is important. In this case, the effect from salts are removed based on the fact that salt is part of the foam formulation. One hypothesis is that the interaction at the liquid interface is controlling the modulus. Indeed, it has been reported that shear modulus is controlled by surface tension, as well as the interactions between surface agents can be probed using shear. This may be a demonstration of the interaction between blood cells and Hm-chitosan, such as gelation or aggregation. Macroscopic cell clusters may be forming when mixing Hm-chitosan with blood, so there may be some interaction between the two components.

### Example 3: The Efficacy of HM-Chitosan in an In Vivo Rat Anterior Epistaxis Model

About 90% of epistaxis cases are anterior epistaxis caused by hemorrhage from vessels in Kiesselbach's plexus in Little's area. Current treatments for epistaxis include embolization, arterial ligation, nasal packing and hemostatic agents such as chitosan, fibrin and floseal. However, there are many drawbacks associated with current treatments, including facial pain, soft tissue necrosis, infections, nasal septum perforation, and cost effectiveness.

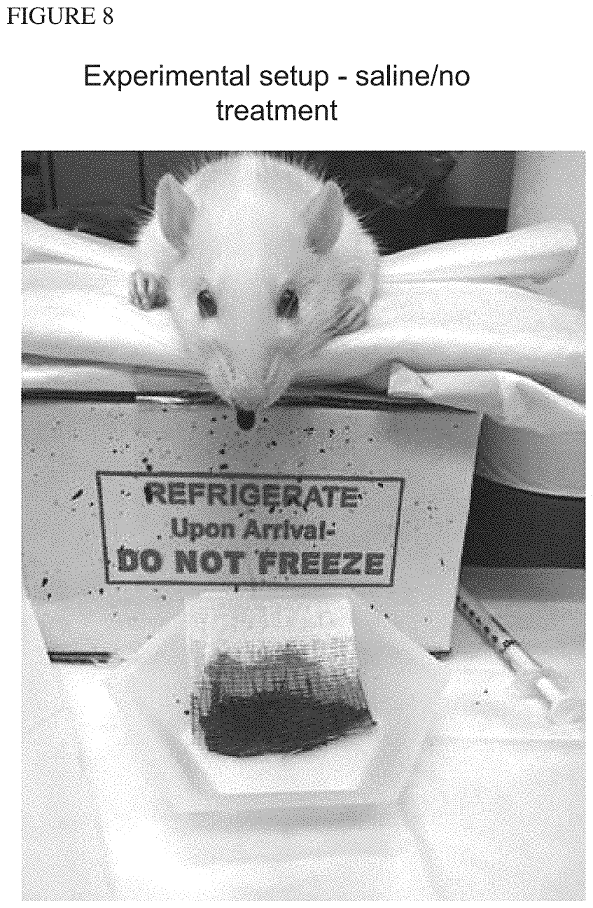

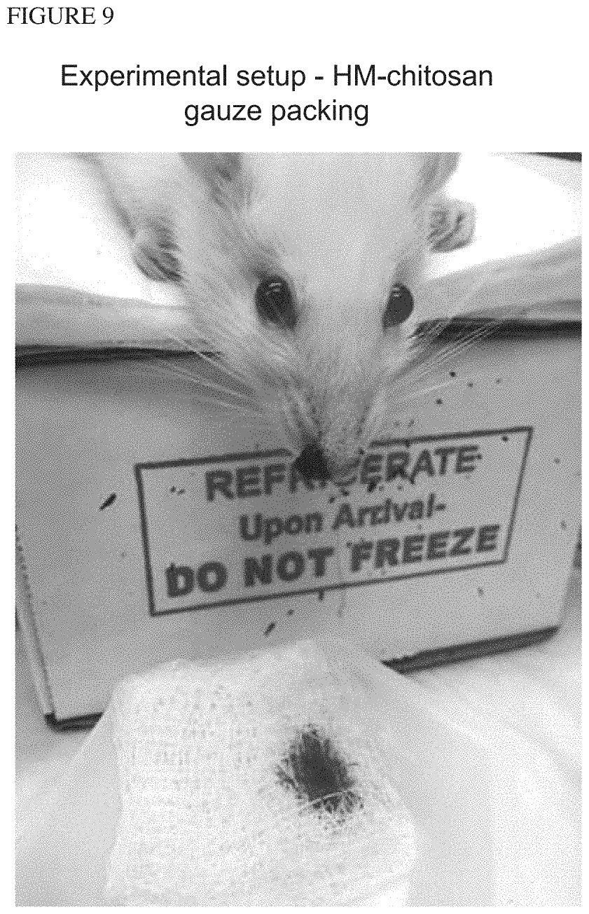

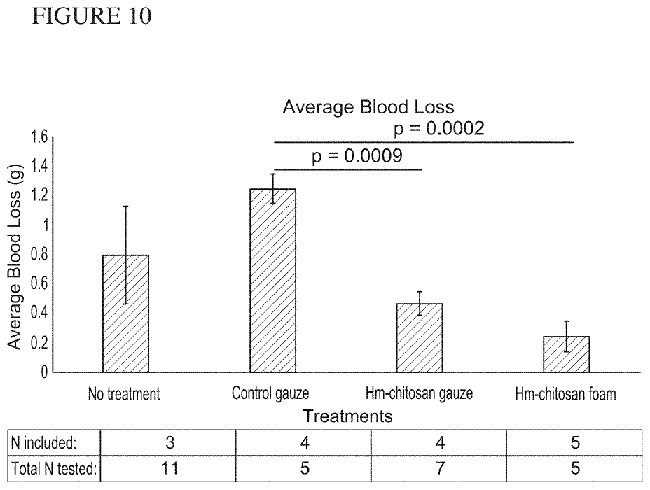

To evaluate the efficacy of HM-chitosan in vivo, a rat anterior epistaxis model was used that enabled the measurement of blood loss and the time needed to reach hemostasis (FIG. 8). In this model, control rats included rats that did not receive any treatment, rats that only received a saline solution (FIG. 9), and rats that received a gauze pack only. The treatment rats included rats that were treated with HM-chitosan foam, rats that were treated with HM-chitosan gauze packing (FIG. 10), or rats that were treated with only chitosan packing (FIG. 8). In this experiment, the rats were initially anesthetized with a combination of ketamine and xylaxine, which was then followed up with intravenous (IV) administration of heparin (640 IU/kg). The rats then received a 2 mm surgical punch, and measurements were then taken to analyze blood loss and the time needed to reach hemostasis. A total of 28 were tested in this model, although 12 were excluded based on the criteria.

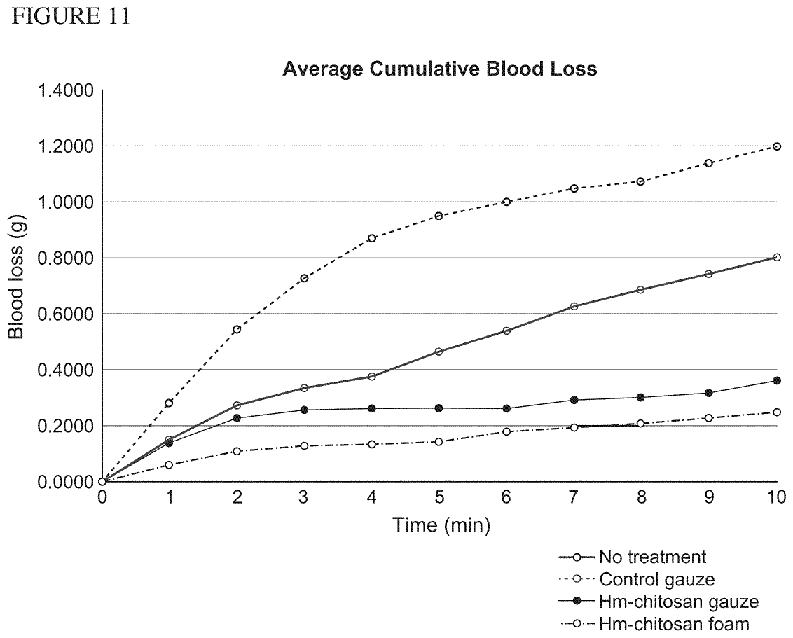

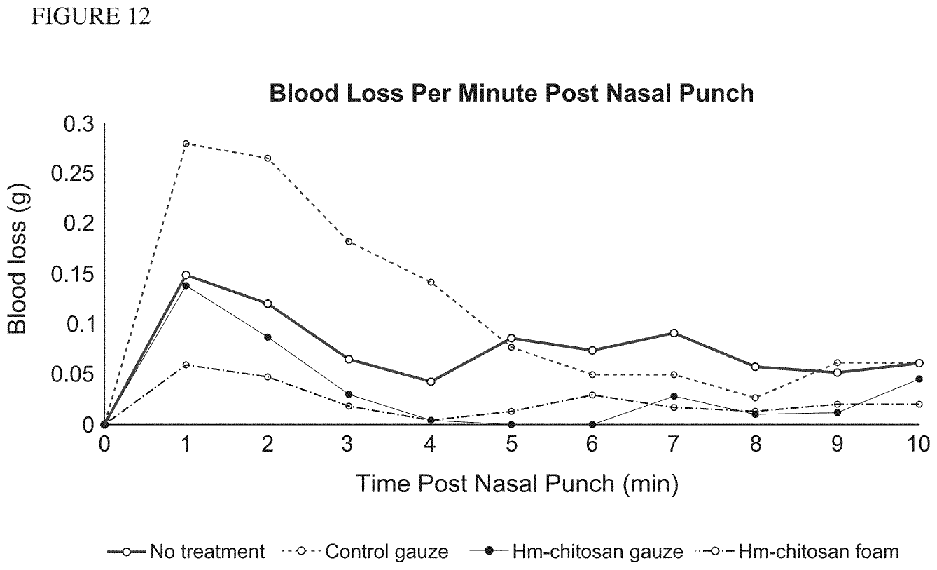

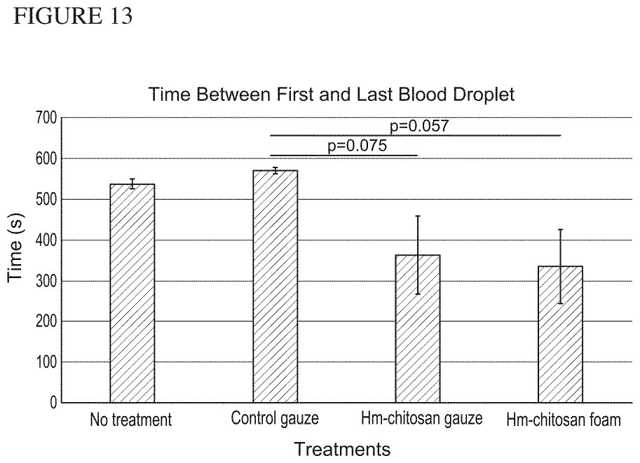

Results demonstrated that rats treated with either the hm-chitosan gauze or the hm-chitosan foam experienced a significant decrease in blood loss compared to either the no treatment control rats or control gauze rats (FIGS. 10 and 11). In addition, rats treated with either the hm-chitosan gauze or the hm-chitosan foam had reduced blood loss per minute post nasal punch when compared to either the no treatment control rats or control gauze rats (FIG. 12). The time needed to reach the first blood droplet was shorter for the rats treated with either the hm-chitosan gauze or the hm-chitosan foam compared to either the no treatment control rats or control gauze rats (FIG. 13). Furthermore, the time needed for total hemostasis was significantly lower for the rats treated with either the hm-chitosan gauze or the hm-chitosan foam compared to either the no treatment control rats or control gauze rats.

1. A hemostat product comprising a container having at least two compartments each containing a releasable, flowable product: wherein a first compartment comprises a solution of a acidified hydrophobically-modified polymer that gels in the presence of blood, and a second compartment comprises a solution of a bicarbonate or carbonate salt at an alkaline pH and optionally a negatively-charged polymer.

2. The product of claim 1, wherein the modified polymer is amphiphilic.

3. The product of claim 2, wherein the polymer is a modified chitosan.

4. The product of claim 3, wherein the polymer is chitosan having a level of deacetylation of from about 40 to about 90%.

5. The product of claim 3 or 4, wherein the polymer has from about 10% to about 50% of functional groups occupied by a hydrophobic group.

6. The product of claim 5, wherein the modified polymer has about 5 to about 100 moles of hydrophobic group per mole of polymer.

7. The product of claim 6, wherein the molecular weight of the polymer is from about 40,000 to about 500,000 Daltons.

8. The product of claim 7, wherein the hydrophobic groups are selected from linear, branched, or cyclic hydrocarbon groups.

9. The product of claim 8, wherein the hydrophobic groups comprise saturated hydrocarbons, which are optionally acyl groups.

10. The product of claim 9, wherein the hydrophobic groups comprise unsaturated, aromatic, and/or polyaromatic hydrocarbon.

11. The product of any one of claims 8 to 10, wherein the hydrophobic groups each have from 6 to about 36 carbon atoms.

12. The product of claim 11, wherein the hydrophobic groups are independently selected from linear hydrocarbons having from 8 to about 18 carbon atoms.

13. The product of any one of claims 1 to 12, wherein the modified polymer is an aqueous formulation.

14. The product of any one of claims 1 to 13, wherein the modified polymer is present at 0.1 to about 2% by weight of the solution in the first compartment.

15. The product of any one of claims 1 to 14, wherein the modified polymer is formulated at a pH of from 2.5 to 4.5.

16. The product of claim 15, wherein the modified polymer is formulated with lactic acid, ascorbic acid, or citric acid.

17. The product of any one of claims 1 to 16, wherein the second compartment comprises a solution comprising sodium bicarbonate.

18. The product of claim 17, wherein the bicarbonate solution is around 0.1 wt % to about 1 wt %.

19. The product of claim 17, wherein the bicarbonate solution is from about 0.2M to about 0.5M sodium bicarbonate.

20. The product of any one of claims 1 to 19, wherein the bicarbonate or carbonate salt is formulated at pH 8 to 10.

21. The product of claim 20, further comprising a negatively-charged polymer in the second compartment.

22. The product of claim 20, wherein the negatively-charged polymer is alginate, pectin, dextran, carboxymethylcellulose, or xanthan gum.

23. The product of claim 20, wherein the alginate is hydrophobically modified.

24. The product of any one of claims 20 to 23, wherein the container is a spray bottle, optionally with a propellant, or the container is a syringe.

25. The product of claim 24, wherein releasing the contents results in mixing of the releasable, flowable products.

26. The product of claim 25, wherein combination of the flowable products results in production of CO₂, and foaming of the hydrophobically-modified polymer composition.

27. A method for treating a bleeding wound, comprising: releasing contents of the product of any one of claims 1 to 26 on said bleeding wound.

28. The method of claim 27, wherein the wound is an internal wound or a wound in a body cavity.

29. The method of claim 28, wherein the bleeding wound is epistaxis, and the method comprises releasing contents of the container into one or more nostrils.

30. The method of claim 29, wherein the resulting foam is applied to the nasal cavity through a conical applicator, or is attached to a substrate that is removed from the nostril once the foam is applied.

31. The method of claim 29 or 30, wherein the resulting foam allows movement of gases through the polymer material.

32. The method of any one of claims 27 to 31, wherein the polymer provides hemostasis within about 2 minutes, or within about 1 minute.

33. A method for treating epistaxis, comprising: applying a solution of a hydrophobically modified polymer into a nasal cavity exhibiting epistaxis.

34. The method of claim 33, wherein the modified polymer is amphiphilic.

35. The method of claim 33, wherein the polymer is chitosan, alginate, or cellulosic biopolymer.

36. The method of claim 35, wherein the polymer is chitosan having a level of deacetylation of from about 40 to about 90%.

37. The method of claim 35 or 36, wherein the polymer has from about 10% to about 50% of functional groups occupied by a hydrophobic group.

38. The method of claim 37, wherein the modified polymer has about 5 to about 100 moles of hydrophobic group per mole of polymer.

39. The method of claim 38, wherein the molecular weight of the polymer is from about 40,000 to about 500,000 Daltons.

40. The method of claim 39, wherein the hydrophobic groups are selected from linear, branched, or cyclic hydrocarbon groups.

41. The method of claim 40, wherein the hydrophobic groups comprise linear, saturated hydrocarbons, which are optionally acyl groups.

42. The method of claim 41, wherein the hydrophobic groups comprise unsaturated, aromatic, and/or polyaromatic hydrocarbon.

43. The method of any one of claims 33 to 42, wherein the hydrophobic groups each have from 6 to about 36 carbon atoms.

44. The method of claim 43, wherein the hydrophobic groups are linear hydrocarbons having from about 12 to about 18 carbon atoms on average.

45. The method of claim 44, wherein the hydrophobic groups are linear hydrocarbons having independently 12 or 18 carbon atoms.

46. The method of any one of claims 33 to 45, wherein the modified polymer is an aqueous formulation.

47. The method of any one of claims 33 to 46, wherein the modified polymer is present at 0.1 to about 2% by weight.
